(19) United States
(12) Patent Application Publication
(10) Pub. No.: US 2024/0376102 A1
(43) Pub. Date: Nov. 14, 2024

(54) AN IMPROVED PROCESS FOR THE PREPARATION OF RUXOLITINIB PHOSPHATE

(71) Applicant: NATCO PHARMA LIMITED, Hyderabad (IN)

(72) Inventors: Satya Srinivas HANUMARA, Hyderabad (IN); Shankar Reddy BUDIDETI, Hyderabad (IN); Srinivasa Krishna KONDURI, Hyderabad (IN); Sunitha KOTHAMUNIREDDY GARI, Hyderabad (IN); Raji Reddy SOLIPURAM, Hyderabad (IN); Jayakrishna KASUGANTI, Hyderabad (IN); Ankama Nayudu ANNADASU, Hyderabad (IN); Venkateswarlu BANDLA, Hyderabad (IN); Lakshmana Viswa Venkata Pavan Kumar MADDULA, Hyderabad (IN); Pulla Reddy MUDDASANI, Hyderabad (IN); Venkaiah Chowdary NANNAPANENI, Hyderabad (IN)

(21) Appl. No.: 18/692,678

(22) PCT Filed: Sep. 16, 2022
(86) PCT No.: PCT/IN2022/050830

§ 371 Date: Mar. 15, 2024

(30) Foreign Application Priority Data
Sep. 18, 2021 (IN) 202141042309

(51) Int. Cl. C07D 471/04; C07D 231/16; C07F 5/02

(52) U.S. Cl. CPC C07D 471/04; C07D 231/16; C07F 5/025

(57) ABSTRACT

The present invention relates to an improved process for the preparation of Ruxolitinib phosphate (1) using novel intermediate compound of Formula (14). The present invention also relates to a process for the preparation of intermediate compound of Formula (14).

### FIELD OF THE INVENTION

The present invention relates to process for the preparation of Ruxolitinib Phosphate of formula (1).

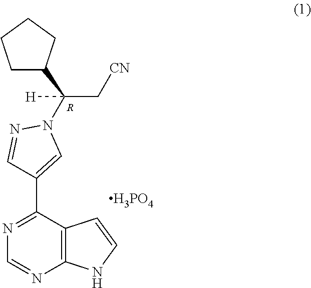

### BACKGROUND OF THE INVENTION

Ruxolitinib phosphate is a heteroaryl-substituted pyrrolo[2,3-d]pyrimidine, also known as (R)-3-(4-(7H-pyrrolo[2,3d]pyrimidin-4-yl)-1H-pyrazol-1-yl)-3-cyclopentylpropanenitrile phosphate is used to treat myelofibrosis (a cancer of the bone marrow in which the bone marrow is replaced by scar tissue and causes decreased blood cell production). It is also used to treat polycythaemia Vera (PV; a slow growing cancer of the blood in which the bone marrow makes too many red blood cells) in people who could not be treated successfully with hydroxyurea. Ruxolitinib is also used to treat graft versus host disease (GVHD; a complication of hematopoietic stem-cell transplant [HSCT; a procedure that replaces diseased bone marrow with healthy bone marrow]) in adults and children 12 years of age and older who were treated unsuccessfully with steroid medications.

Ruxolitinib inhibited cytokine-induced STAT3 phosphorylation in whole blood from healthy subjects and myelofibrosis patients. Ruxolitinib resulted in maximal inhibition of STAT3 phosphorylation 2 hours after dosing which returned to near baseline by 8 hours in both healthy subjects and myelofibrosis patients, indicating no accumulation of either parent or active metabolites.

Ruxolitinib phosphate has been approved in the US and Europe for the treatment of myelofibrosis and for the treatment of polycythemia vera. Ruxolitinib is currently in clinical trials for the treatment of graft-versus-host disease and other conditions.

U.S. Pat. No. 7,598,257 discloses a process (Scheme-1) for the preparation of Ruxolitinib (1a) by reacting cyclopentane carbaldehyde (2) with diethyl cyanomethylphosphonate (3) in the presence of potassium tert-butoxide in THF for about 64 h. Later, the reaction mixture was partitioned between diethylether and water. The organic layer is distilled off completely and co-distilled with ethyl acetate and subsequent washings with brine, drying over anhydrous sodium sulfate and evaporation to obtain (2E) and (2Z)-3-Cyclopentylacrylonitrile (4). Compound of formula (4) is reacted with 4-(1H-pyrazol-4-yl)-7-[2-(trimethylsilyl)ethoxy]methyl-7H-pyrrolo[2,3-d]-pyrimidine (5) in the presence of DBU in acetonitrile. Later, acetonitrile is distilled off completely, diluted with ethylacetate, washing with 1N HCl and separated the ethylacetate layer. The organic layer is washed with brine and drying of anhydrous sodium sulfate and concentrated. The resulting crude product is purified by silica gel choromatography and further evaporative with the help of ethanol to obtain SEM protected racemic mixture of Ruxolitinib (6). Later, SEM protected racemic mixture of Ruxolitinib (6) is subjected to preparative HPLC in ethylacetate-n-hexane to separate required (R) isomer (7). Finally, SEM protected (R)-isomer is deprotected in the presence of trifluoroacetic acid in dichloromethane followed by distillation of TFA, treatment with ethylenediamine, evaporation, extraction with ethyl acetate and evaporation to afford crude Ruxolitinib free base (1a). Crude Ruxolitinib is further purified by flash chromatography followed by preparative HPLC to afford Ruxolitinib free base (1a).

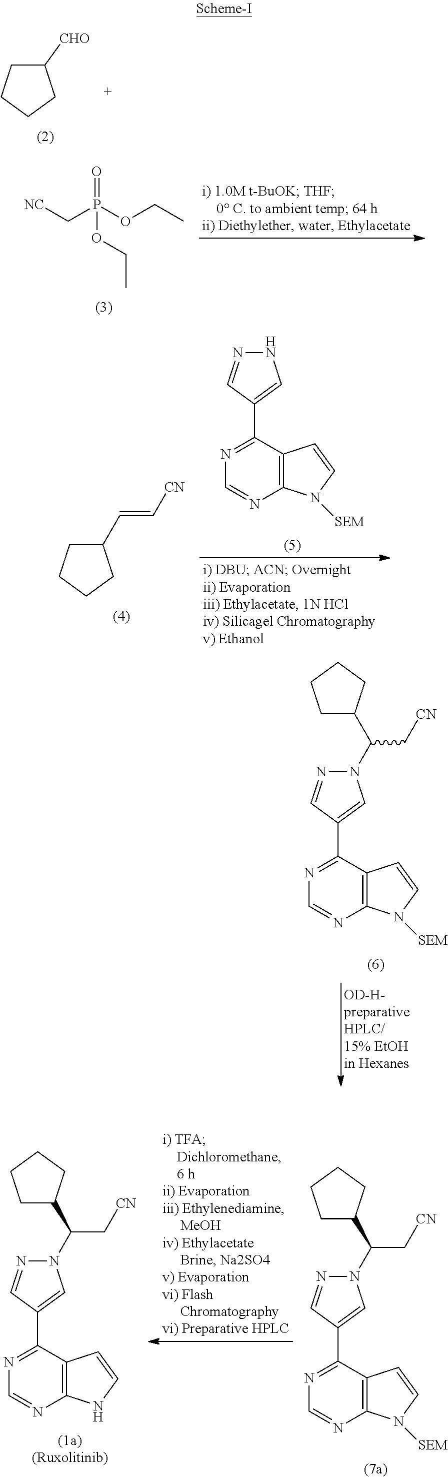

In U.S. Pat. No. 8,410,265, a process for the preparation of Ruxolitinib phosphate is disclosed. According to process disclosed in the patent (Scheme-2), Boronate ester (8) is reacted with SEM-protected derivative (9) in the presence of palladium catalyst and potassium carbonate as base to afford pyrazole (5). Pyrazole (5) is reacted with (2E) and (2Z)-3-Cyclopentylacrylonitrile (4) in the presence of DBU in acetonitrile followed by purification using silicagel chromatography to afford SEM protected racemic mixture of Ruxolitinib (6). (R)-enantiomer of SEM protected racemic mixture of Ruxolitinib (6) is separated by SMB purification technique. Later, (R)-enantiomer of SEM protected Ruxolitinib (7) is reacted with lithium tetrafluoroborate followed by treatment with aqueous ammonia to afford crude Ruxolitinib base (1a) which is further purified by flash chromatography. Finally, purified Ruxolitinib base (1a) is transformed to Ruxolitinib phosphate salt (1) using phosphoric acid in IPA.

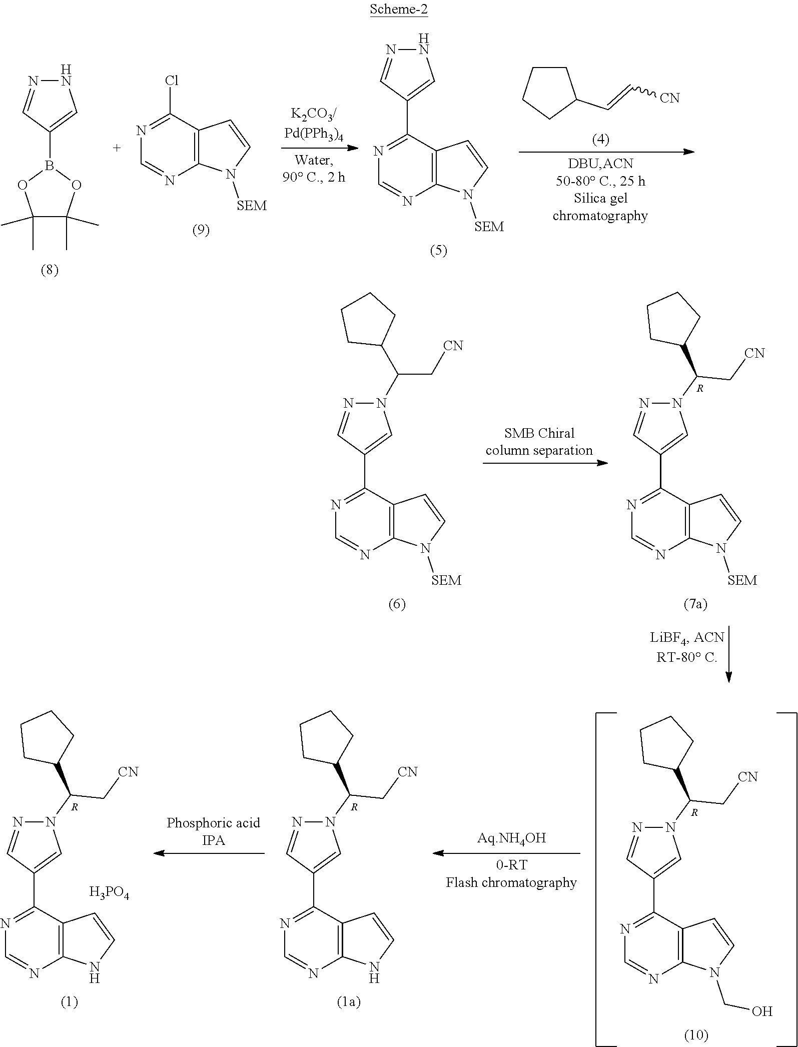

In spite of having variety of methods for the preparation of Ruxolitinib phosphate (1), there is still a need to develop commercially viable process to meet the demand for the treatment of patients with myelofibrosis. Based on this prevailing situation in the world, we have focused to work on developing commercially viable process for Ruxolitinib phosphate (1) as it has less side effects in treating cancer patients.

The inventors of the present invention have developed a simple, and novel process for the preparation of Ruxolitinib phosphate (1) and its intermediate (14). The present process is cost effective and scalable on commercial scale.

### Objective of the Invention

The main objective of the present invention is to provide a simple, cost-effective for the preparation of Ruxolitinib phosphate (1) and its intermediate (14) with high purity and overall yield.

Another objective of the present invention is to provide a process, which is simple, economical and suitable for industrial scale up.

### SUMMARY OF THE INVENTION

One aspect of the present invention is to provide a novel process for the preparation of Ruxolitinib phosphate (1) and its intermediate (14) as depicted below in scheme-3.

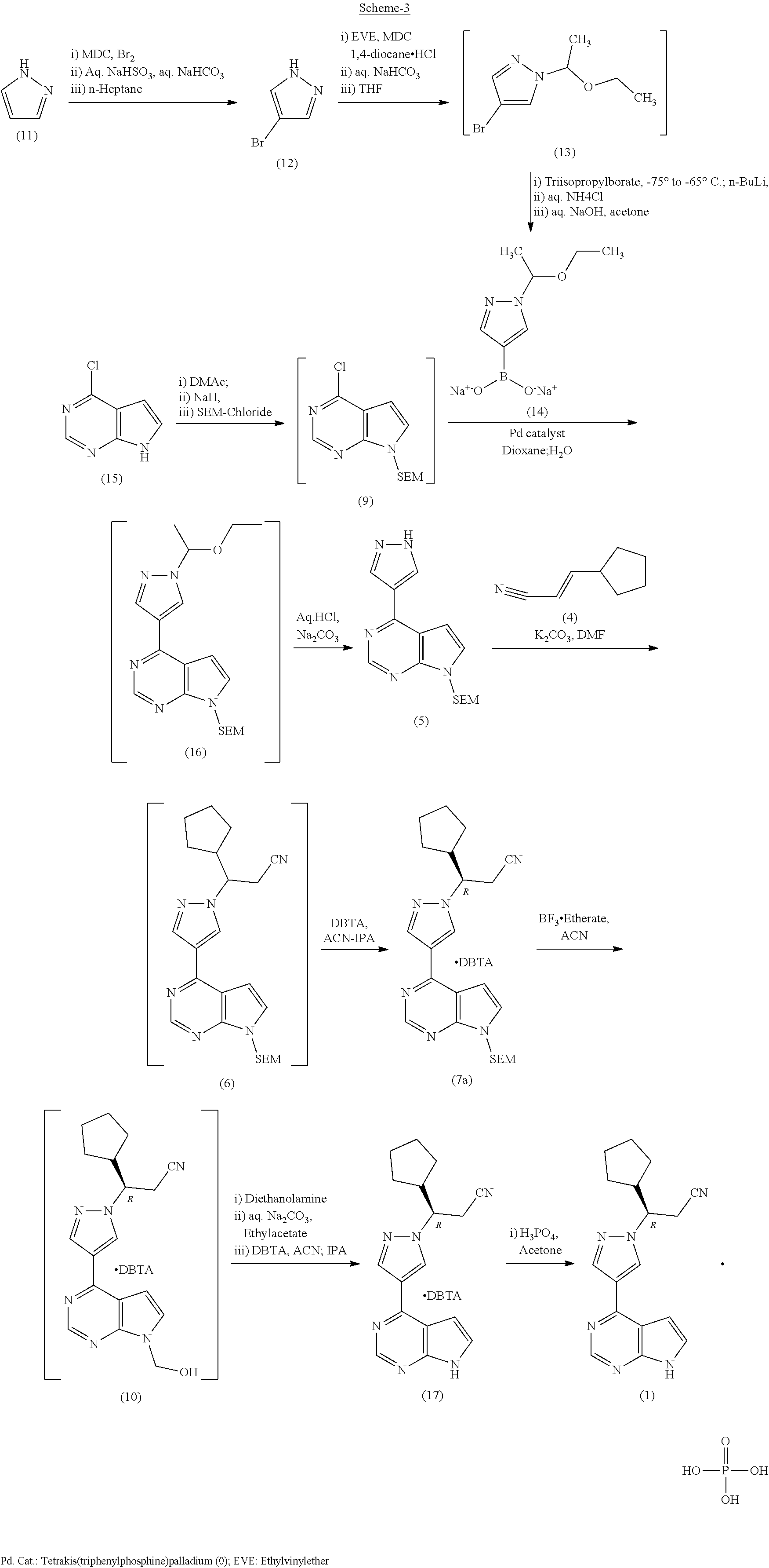

Another aspect of the present invention is to provide a process for process for the preparation of compound of formula (14) as depicted below in scheme-4.

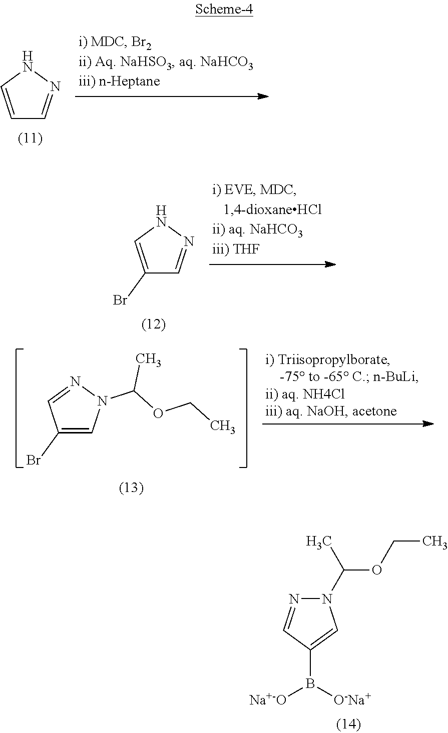

Another aspect of the present invention is to provide the following novel compound (14) used in the process for the preparation of Ruxolitinib phosphate (1).

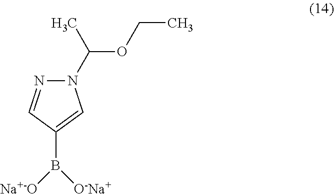

Another aspect of the present invention is to provide a process for recovery and recycling of unwanted (S)-isomer of formula (7b) to formula (5) as depicted below in scheme-5.

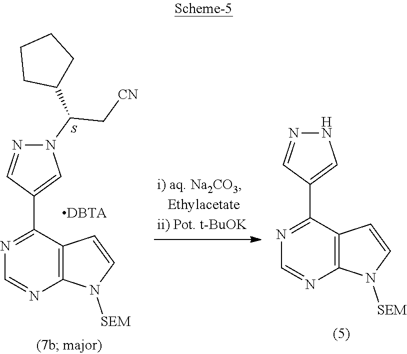

Another aspect of the present invention provides a novel process for preparation of novel compound of formula (14)

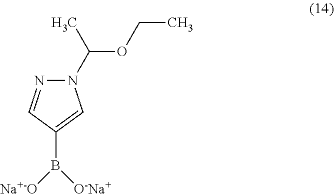

comprising the steps of:
a) reacting Pyrazole (11)

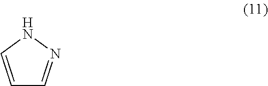

With bromine in the an organic solvent to afford compound of formula (12)

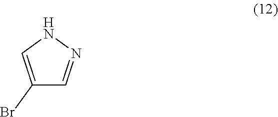

b) reacting compound of formula (12) with ethyl vinyl ether in an organic solvent in the presence of catalytic amount of acid reagent to afford compound of formula (13)

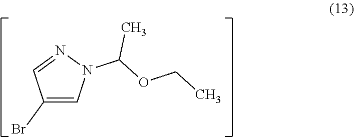

c) reacting compound of formula (13) with triisopropylborate in the presence of n-Butyl lithium in an organic solvent followed by reaction with aqueous sodium hydroxide and isolation from an organic solvent to afford compound of formula (14)

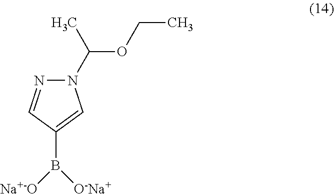

Another aspect of the present invention is to provide process for the preparation of Ruxolitinib phosphate (1) involving novel compound of formula (14)

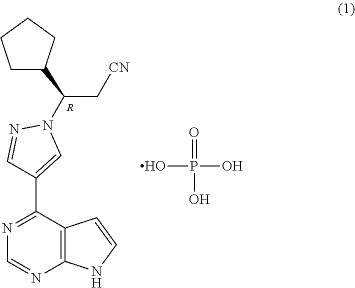

comprising the steps of:
a) reacting compound of formula (15)

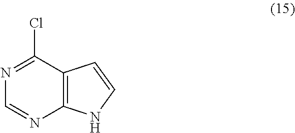

with SEM-chloride in the presence of base in organic solvent to afford compound of formula (9)

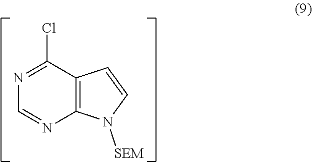

b) reacting compound of formula (9) with novel compound of formula (14)

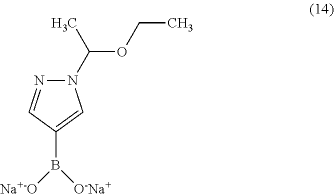

in the presence of palladium catalyst in an aqueous organic solvent to afford compound of formula (16)

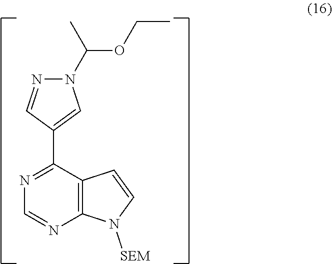

c) reacting compound of formula (16) with acid followed by treatment with aqueous base to afford compound of formula (5)

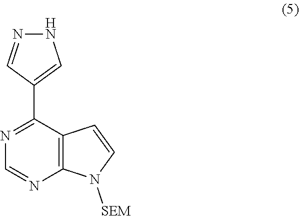 [SMILES: C[Si](C)(C)CCOCn1ccc2c(-c3cn[nH]c3)ncnc21]

d) reacting compound of formula (5) with compound of formula (4) in the presence of base and organic solvent

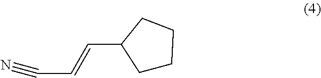

to afford compound of formula (6)

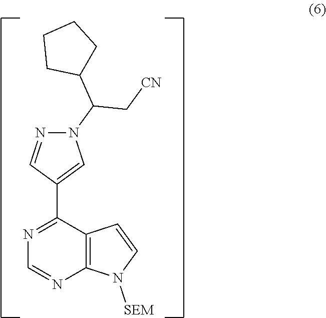

e) reacting compound of formula (6) with Di-Benzoyl-D-Tartaric acid followed by crystallization in an organic solvent mixture to afford compound formula (7a)

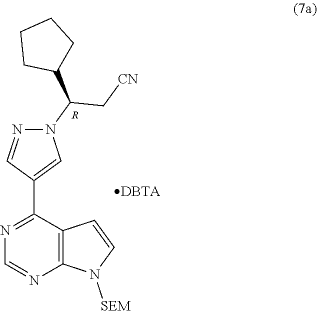

f) reacting compound of formula (7a) with Boron trifluoride etherate in an organic solvent to afford compound of formula (10)

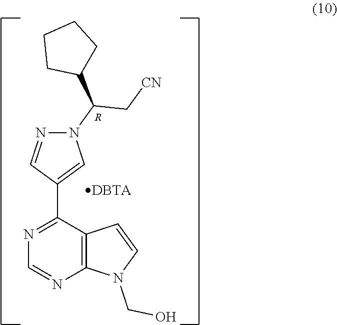

g) reacting compound of formula (10) with an organic base followed by treatment with Di-Benzoyl-D-Tartaric acid and purification to afford compound of formula (17)

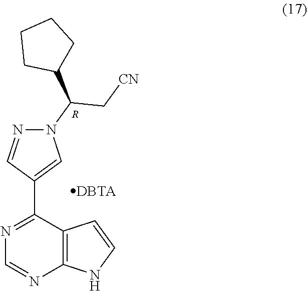

h) reacting compound of formula (17) with phosphoric acid in an organic solvent to afford compound of formula (1)

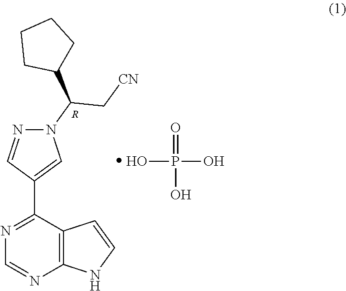

i) purification of Ruxolitinib phosphate formula (1) with an organic solvent or solvent mixture to afford chemically and chirally pure Ruxolitinib phosphate of formula (1)

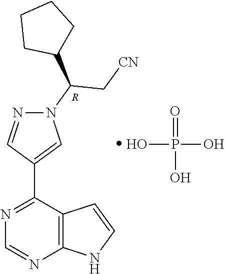

The present invention also provides process for recovery of compound of formula (5)
a) mother liquors of compound of formula (7a) containing majorly compound of formula (7b) is treating with aqueous base followed by extraction with an organic solvent

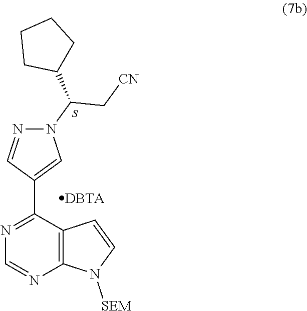

b) followed by treatment with base to afford compound of formula (5)

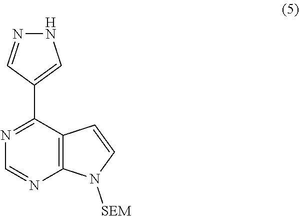 [SMILES: C[Si](C)(C)CCOCn1ccc2c(-c3cn[nH]c3)ncnc21]

which is further used in the process for the preparation of Ruxolitinib phosphate of formula (1)

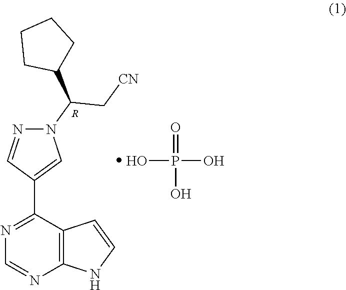

### BRIEF DESCRIPTION OF THE DRAWINGS

FIG. 1 shows a representative X-ray powder diffraction (XRPD) pattern of crystalline Ruxolitinib phosphate salt of Formula-I.

FIG. 2 shows a representative differential scanning calorimetry (DSC) thermogram of crystalline Ruxolitinib phosphate salt of Formula-I.

### DETAILED DESCRIPTION OF THE INVENTION

The present invention provides a simple and cost-effective process for the preparation of compound of formula (1) involving novel intermediate of compound of formula (14), comprising steps of:
a) In step (a) of the present invention, reacting Pyrazole (11) with bromine in an organic solvent to afford compound of formula (12)
b) In step (b) of the present invention, reacting compound of formula (12) with ethyl vinyl ether in an organic solvent in the presence of catalytic amount of acid reagent to afford compound of formula (13)
c) In step (c) of the present invention, reacting compound of formula (13) with triisopropylborate in the presence of n-Butyl lithium in an organic solvent followed by reaction with aqueous sodium hydroxide and isolation from an organic solvent to afford compound of formula (14)

In step (a) of the present invention, bromination of pyrazole (11) in an organic solvent selected from dichloromethane, chloroform, acetonitrile, water or mixture thereof.

In step (a) of the present invention, the temperature at which bromination carried out at −20° to 40° C. preferably 20-35° C.

In step (a) of the present invention, 4-bromopyrazole (12) is isolated from organic solvent selected from dichloromethane, n-heptane, hexanes, n-hexane, methyl t-butyl ether, water or mixture thereof.

In step (b) of the present invention, 4-bromopyrazole (12) is reacted with ethyl vinyl ether in an organic solvent selected from dichloromethane, chloroform, methanol, isopropylalchol, 1,4-dioxane, methyl t-butyl ether, tetrahydrofuran or any other suitable organic solvent.

In step (b) of the present invention, acid catalyst used is selected from, gaseous HCl in combination with methanol, ethanol, ethyl acetate, 1,4-dioxane, isopropyl alcohol etc.

In step (b) of the present invention, after completion of reaction, the reaction mass is treated with aqueous base selected from aqueous sodium carbonate, aqueous sodium bicarbonate, aqueous potassium carbonate, aqueous potassium bicarbonate, aqueous sodium hydroxide or any other suitable aqueous base.

In step (b) of the present invention, the resulting product (13) is directly used in the next step without further isolation.

In step (c) of the present invention, the compound of formula (13) is reacted with trimethylborate, triisopropylborate in the presence of base selected from n-butyl lithium, organo magnesium halide in an organic solvent selected from tetrahydrofuran, 1,4-dioxane, methyl t-butyl ether, 2-methyl THF, cyclopentyl methyl ether etc preferably tetrahydrofuran.

In step (c) of the present invention, the temperature at which the reaction is carried out between −85° C. to 35° C. preferably at −80° C. to −40° C.

In step (c) of the present invention, after completion of reaction, the reaction mass is quenched with aqueous ammonium chloride, water, acetic acid, aqueous HCl etc.

In step (c) of the present invention, after separation and concentration of organic layer, the resulting residue is treated with aqueous sodium hydroxide in an organic solvent selected from acetone, methanol, acetonitrile, isopropyl alcohol, tetrahydrofuran, ethyl acetate, 1,4-dioxane, methyl t-butyl ether, isopropyl ether or any other suitable organic solvent to afford compound of formula (14).
a) In step (a) of the present invention, reacting compound of formula (15) with SEM-chloride in the presence of base in organic solvent to afford compound of formula (9)
b) In step (b) of the present invention, reacting compound of formula (9) with novel compound of formula (14) in the presence of palladium catalyst in an aqueous organic solvent to afford compound of formula (16)
c) In step (c) of the present invention, reacting compound of formula (16) with acid followed by treatment with aqueous base to afford compound of formula (5)
d) In step (d) of the present invention, reacting compound of formula (5) with compound of formula (4) in the presence of base and organic solvent to afford compound of formula (6)
e) In step (e) of the present invention, reacting compound of formula (6) with Di-Benzoyl-D-Tartaric acid followed by crystallization in an organic solvent mixture to afford compound formula (7a)
f) In step (f) of the present invention, reacting compound of formula (7a) with Boron trifluoride etherate in an organic solvent to afford compound of formula (10)
g) In step (g) of the present invention, reacting compound of formula (10) with an organic base followed by treatment with Di-Benzoyl-D-Tartaric acid and purification to afford compound of formula (17)
h) In step (h) of the present invention, reacting compound of formula (17) with phosphoric acid in an organic solvent to afford compound of formula (1)
i) In step (i) of the present invention, purification of Ruxolitinib phosphate formula (1) with an organic solvent or solvent mixture to afford chemically and chirally pure Ruxolitinib phosphate of formula (1).

In step (a) of the present invention, compound of formula (15) is reacted with SEM-chloride in the presence of base selected form potassium carbonate, sodium carbonate, sodium hydride, cesium carbonate, potassium t-butoxide etc.

In step (a) of the present invention, the organic solvent selected from N,N-dimethylformamide, N,N-dimethylacetamide, tetrahydrofuran, dimethylsulfoxide, N-Methylpyrrolidone, 1,4-dioxane or mixture thereof.

In step (a) of the present invention, after completion of reaction, the reaction mass is quenched with aqueous organic solvent selected from N,N-dimethylformamide, N,N-dimethylacetamide, tetrahydrofuran, dimethylsulfoxide, N-Methylpyrrolidone, 1,4-dioxane, methanol, ethanol, isopropyl alcohol or water.

In step (a) of the present invention, after quenching of the reaction mass, the product is extracted with organic solvent selected from dichloromethane, tetrahydrofuran, ethyl acetate, 1,4-dioxane, methyl t-butyl ether, isopropyl ether or any other suitable organic solvent.

In step (a) of the present invention, after separation and concentration of organic layer, the resulting residue is used in the next step without further isolation.

In step (b) of the present invention, compound of formula (9) is reacted novel compound of formula (14) in the presence of palladium catalyst selected from tetrakis(triphenylphosphine)palladium (0), [1,1′-Bis(diphenylphosphino)ferrocene]dichloropalladium(II), [1,1′-Bis(diphenylphosphino)ferrocene]dichloropalladium(II) DCM complex, Bis(triphenylphosphine)palladium(II) dichloride, palladium acetate, palladium carbon or any other suitable palladium catalyst under inert atmosphere.

In step (b) of the present invention, the solvent is selected from 1,4-dioxane, tetrahydrofuran, water, isopropyl alcohol, ethanol, toluene, N,N-dimethylformamide, N,N-dimethylacetamide or mixture thereof.

In step (b) of the present invention, the temperature at which the reaction is carried out between 50° C. to 110° C.

In step (b) of the present invention, after completion of reaction and filtration of catalyst, the product is extracted with organic solvent selected from ethyl acetate, dichloromethane, toluene, methyl t-butyl ether or any other suitable organic solvent.

In step (c) of the present invention, after concentration of organic layer, the resulting residue is treated with acid selected from aq. HCl, aq. acetic acid or any other suitable acid.

In step (c) of the present invention, the temperature at which acid treatment is carried out between −10° to 35° C.

In step (c) of the present invention, after treatment with acid, the product is extracted with organic solvent selected from ethyl acetate, methyl t-butyl ether, toluene, tetrahydrofuran, dichloromethane or any other suitable organic solvent.

In step (c) of the present invention, after separation and concentration of organic layer, the resulting product is treated with organic solvent selected from acetone, acetonitrile, isopropyl alcohol, n-heptane, n-hexane or hexane followed by purification from solvent mixture selected from isopropyl alcohol, acetonitrile, acetone, water etc.

In step (d) of the present invention, compound of formula (5) is reacted with compound of formula (4) in the presence of base selected from potassium carbonate, sodium carbonate, sodium hydride, cesium carbonate, potassium t-butoxide etc.

In step (d) of the present invention, the organic solvent selected from N,N-dimethylformamide, N,N-dimethylacetamide, tetrahydrofuran, dimethylsulfoxide, N-Methylpyrrolidone, 1,4-dioxane, methanol, ethanol, isopropyl alcohol or water.

In step (d) of the present invention, the temperature at which the reaction is carried out between 0° C. to 100° C.

In step (d) of the present invention, after completion of reaction, the reaction mass is quenched with water followed by extraction with organic solvent selected from dichloromethane, tetrahydrofuran, ethyl acetate, 1,4-dioxane, methyl t-butyl ether, isopropyl ether or any other suitable organic solvent.

In step (e) of the present invention, after separation and concentration, the resulting residue is reacted with Di-Benzoyl-D-Tartaric acid in an organic solvent selected from isopropyl alcohol, acetonitrile, acetone, tetrahydrofuran, 1,4-dioxane, methanol, ethanol, dimethyl sulfoxide or mixture thereof.

In step (e) of the present invention, the temperature at which the DBTA salt formation is carried out between 10° C. to 100° C.

In step (e) of the present invention, the resulting product (7a) is purified from organic solvent selected from isopropyl alcohol, acetonitrile, acetone, tetrahydrofuran, 1,4-dioxane, methanol, ethanol, dimethyl sulfoxide or mixture thereof.

In step (f) of the present invention, the compound of formula (7a) is reacted with boron trifluoride etherate in an organic solvent selected from acetonitrile, acetone, tetrahydrofuran, 1,4-dioxane, or mixture thereof.

In step (f) of the present invention, the temperature at which the reaction is carried out between 10° C. to 45° C.

In step (g) of the present invention, the reaction mass obtained in step (f) is treated with organic base selected from diethanolamine, monoethanolamine, triethanolamine, methylamine etc preferably Diethanolamine.

In step (g) of the present invention, the temperature at which the reaction is carried out between −20° C. to 50° C.

In step (g) of the present invention, after completion of reaction, the reacted mass is quenched with water and extracted with organic solvent selected from ethyl acetate, methyl t-butyl ether, dichloromethane, chloroform, tetrahydrofuran etc.

In step (g) of the present invention, after separation and concentration of solvent, the resulting residue is reacted with Di-Benzoyl-D-Tartaric acid in an organic solvent selected from isopropyl alcohol, acetonitrile, acetone, tetrahydrofuran, 1,4-dioxane, methanol, ethanol, dimethyl sulfoxide or mixture thereof.

In step (g) of the present invention, the temperature at which the DBTA salt formation and isolated is carried out between 10° C. to 100° C.

In step (g) of the present invention, the resulting product is purified by DBTA salt breaking with aqueous base selected from aqueous sodium carbonate, aqueous potassium carbonate etc and making chirally and chemically pure compound of formula (17) using Di-Benzoyl-D-Tartaric acid in an organic solvent selected from isopropyl alcohol, acetonitrile, acetone, tetrahydrofuran, 1,4-dioxane, methanol, ethanol, dimethyl sulfoxide or mixture thereof.

In step (h) of the present invention, chirally and chemically pure compound of formula (17) is converted to Ruxolitinib phosphate of formula (1) with phosphoric acid in an organic solvent selected from acetone, acetonitrile, isopropyl alcohol, dichloromethane, tetrahydrofuran, 1,4-dioxane or any other suitable organic solvent.

In step (i) of the present invention, Ruxolitinib phosphate of formula (1) is purified from organic solvent selected from acetone or isopropyl alcohol to afford chirally and chemically pure Ruxolitinib phosphate of formula (1).

The present invention also provides process for recovery of compound of formula (5)
a) mother liquors of compound of formula (7a) containing majorly compound of formula (7b) is treating with aqueous base followed by extraction with an organic solvent

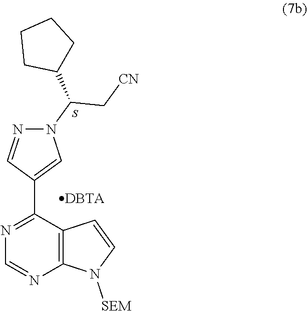

b) followed by treatment with base to afford compound of formula (5)

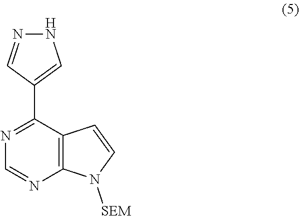 [SMILES: C[Si](C)(C)CCOCn1ccc2c(-c3cn[nH]c3)ncnc21]

which is further used in the process for the preparation of Ruxolitinib phosphate of formula (1)

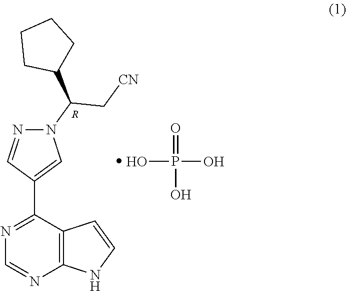

In step (a) of the present invention, compound of formula (7b) recovered from mother liquors of compound of formula (7a) by concentration of solvent and treatment with base selected from aqueous potassium carbonate, aqueous sodium carbonate or aqueous sodium bicarbonate and extraction of compound of formula (7b) with organic solvent selected from ethyl acetate or dichloromethane or methyl t-butyl ether.

In step (b) of the present invention, after completion of reaction, the compound of formula (5) extracted with organic solvent selected from ethyl acetate or dichloromethane.

In step (b) of the present invention, after concentration of solvent, the resulting product of formula (5) is treatment with organic solvent selected from acetone, acetonitrile, isopropyl alcohol, n-heptane, n-hexane or hexane followed by purification from solvent mixture selected from isopropyl alcohol, acetonitrile, acetone, water.

In step (b) of the present invention, the resulting product of formula (5) is used in the preparation of Ruxolitinib phosphate of formula (1) as described in step (g) to step (k).

The present invention also provides a crystalline Ruxolitinib phosphate of Formula-I, characterized by powder X-ray diffraction pattern (PXRD) comprising the peaks at about 4.0, 14.4, 15.8, 20.1, 20.8, 21.7, 24.8, 15.9, 16.5, 25.2 and 26.2°±0.2° 2θ″.

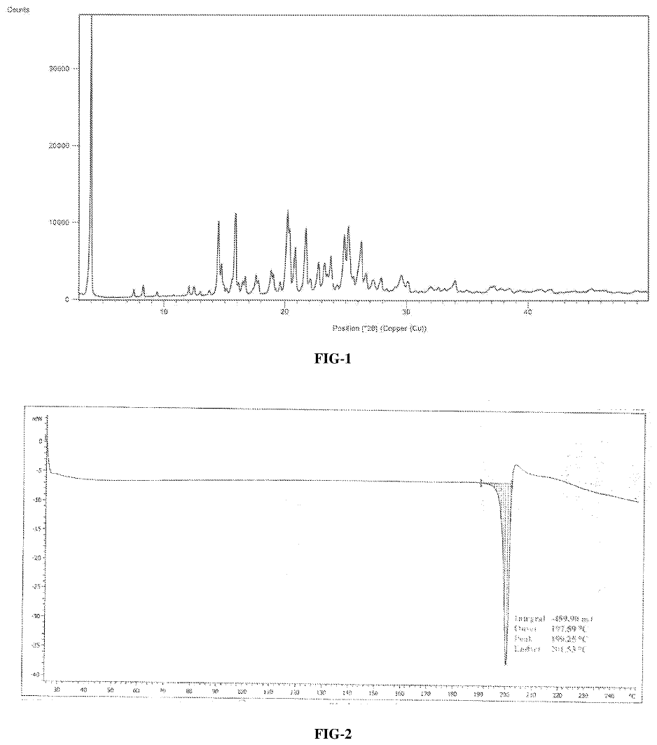

The present invention further provides crystalline Ruxolitinib phosphate characterized in that its powder X-ray powder diffraction pattern is basically the same as that of FIG. 1.

The Differential Scanning Calorimetric (DSC) thermogram of crystalline Ruxolitinib phosphate exhibits single endotherm peak at about 199±5° C. same as that of FIG. 2.

### Advantages of the Present Invention

1. The process of the present invention is successfully produces Ruxolitinib phosphate (1) with simple and commercially viable purification techniques.
2. The process of the present invention is successfully avoids usage of large volumes of organic solvent by developing cost effective process.
3. The process of the present invention for novel compound of formula (14) is cheap and commercially viable.
4. The process of the present invention for compound of formula (16) is successfully avoids additional base during reaction.
5. The process of the present invention for the preparation of compound of formula (10) from compound of formula (7a) is novel.
6. The process of the present invention for Ruxolitinib phosphate of formula (1) involving novel compound of formula (14) is economical and commercially scalable.
7. The process of the present invention produces Ruxolitinib phosphate of formula (1) economical process with high quality.
8. The process of the present invention for Ruxolitinib phosphate of formula (1) from compound of formula (17) is novel and advantageous as corresponding base of compound of formula (17) is thick oil and difficult to handle.

The following examples are provide for illustration purpose only and are not intended to limit the scope of invention.

### EXAMPLES

### Example-01: PREPARATION OF 4-BROMO-1H-PYRAZOLE (12)

Into a mixture of methylene chloride (2.0 L) and Pyrazole (100 g), bromine (246.4 g) was added drop wise at −5 to 0° C. and further maintained at 25-30° C. for ˜24 h. Progress of the reaction was monitored by HPLC analysis. After completion of reaction, the reaction mass was quenched by addition of ˜1.5% aq. sodium bisulfite (1.0 L) and further basified by lot wise addition of sodium bicarbonate (185 g). The lower organic layer was separated, washed with saturated aq. sodium chloride solution (200 mg) and concentrated under vacuum to afford crude 4-Bromo-1H-pyrazole. Finally, crude 4-Bromo-1H-pyrazole was purified and isolated from n-heptane (400 mL) to afford pure 4-Bromo-1H-pyrazole as crystalline solid.

HPLC purity: >99.0%

### Example-02: PREPARATION OF 4-BROMO-1-(1-ETHOXYETHYL)-1H-PYRAZOLE (13)

Into a reaction flask, Ethyl vinyl ether (103.01 g) was added drop wise to a solution of 4-Bromo-1H-pyrazole (12; 175 g) in methylene chloride (1.4 L) at 10-15° C. Later, to the reaction mass, 1,4-dioxane-hydrogen chloride (17.5 mL) was added lot wise at 0-10° C. and maintained at 10-15° C. for about 4 h. Progress of the reaction was monitored by HPLC. After completion of reaction, the reaction mass was washed with aq. sodium bicarbonate (875 mL), water (875 mL), aq. NaCl (875 mL) and separated. The organic layer was concentrated and co-distilled with THF under vacuum to afford 4-Bromo-1-(1-ethoxyethyl)-1H-pyrazole (13) as thick oily syrup. 4-Bromo-1-(1-ethoxyethyl)-1H-pyrazole (13) was directly used in the next stage without further analysis.

### Example-03: PREPARATION OF DISODIUM [1-(1-ETHOXYETHYL) PYRAZOL-4-YL]-DIOXIDO-BORANE (14)

4-Bromo-1-(1-ethoxyethyl)-1H-pyrazole (13; 250 g) was reacted with triisopropylborate (250 g) in tetrahydrofuran (2.5 L) in the presence of n-butyl lithium (1.0 L; 1.6M in hexane) at −70° to −65° C. Progress of the reaction was monitored by HPLC. After completion of reaction, the reaction mass was treated with aq. ammonium chloride (1.25 L) and separated the organic layer. Later, the organic layer was concentrated to dryness under vacuum and the resulting syrupy compound was reacted with ˜50% aq. sodium hydroxide (220 mL) in acetone (2.0 L). The resulting sodium salt (14) was filtered and dried under vacuum to afford Disodium [1-(1-ethoxyethyl)pyrazol-4-yl]-dioxido-borane (224.0 g; 86.1% by theory; 14) as off-white to light brown hygroscopic solid.

HPLC purity: >99.5%

Mol. Formula: C₇H₁₄BN₂O₃·2Na

¹HNMR (δ ppm; CD₃OD; 400 MHz): 7.46 (s, 1H), 7.42 (s, 1H), 5.42-5.47 (q, 1H), 1.61-1.62 (d, 3H), 3.35-3.42 (m, 1H), 3.15-3.23 (m, 1H), 1.04-1.08 (t, 3H)

¹³CNMR (δ ppm; CD₃OD; 100 MHz): 145.09, 125.90, 131.99, 87.18, 22.11, 64.43, 15.18

FTIR (λ cm⁻¹): 2985; 2945 (aliphatic —C—H str.); 1377 (—B—O str.); 1113, 1061 (—C—O str.)

Mass: 184.42 [M+H]⁺ (free boronic acid)

Sodium content: 19.42% (w/w by IC method corresponds to disodium salt of formula (14))

### Example-04: PREPARATION OF 4-CHLORO-7-[[2-(TRIMETHYLSILYL) ETHOXY] METHYL]-7H-PYRROLO[2,3-D]PYRIMIDINE (9)

4-Chloro-7H-pyrrolo [2,3-d]pyrimidine (150 g; 15) was protected with SEM-Cl (213.4 g) in the presence of sodium hydride (46.5 g; 65% in paraffin oil) in N,N-dimethylacetamide (1.5 L) at −15 to −5° C. After completion of the reaction monitored by HPLC, reaction mass was quenched with DM water and extracted with MTBE. The organic layer was washed with DM water. Finally, organic layer was treated with activated carbon, filtered through celite bed, concentrated under vacuum to afford 4-chloro-7-[[2-(trimethylsilyl)ethoxy]methyl]-7H-pyrrolo[2,3-d]pyrimidine (9). Later it is used in the next stage as such without further purification.

### Example-05: PREPARATION OF [4-[1-(1-ETHOXYETHYL)-]H-PYRAZOL-4-YL]-7-[[2-(TRI METHYLSILYL)ETHOXY] METHYL]-7H-PYRROLO[2,3-D]PYRIMIDINE (16)

The above concentrated mass (9) was reacted with disodium [1-(1-ethoxyethyl)pyrazol-4-yl]-dioxido-borane (301.5 g; 14) in the presence of Tetrakis(triphenylphosphine) palladium(0) (3.38 g) in 1,4-dioxane (2.13 L) and DM water (0.48 L) at 85-90° C. under nitrogen atmosphere. Progress of the reaction was monitored by HPLC. After completion of the reaction, the reaction mass was treated with activated carbon and filtered. To the filtrate, ethyl acetate and DM water were added and organic layer was separated. Organic layer was washed with aqueous sodium chloride solution and concentrated under vacuum at below 45-50° C. to yield [4-[1-(1-Ethoxyethyl)-1H-pyrazol-4-yl]-7-[[2-(trimethylsilyl)ethoxy]methyl]-7H-pyrrolo[2,3-d] pyrimidine (16) as oily product. The compound of formula (16) was used in the next stage as such without further purification.

### Example-06: PREPARATION OF 4-(1H-PYRAZOL-4-YL)-7-[[2-(TRIMETHYLSILYL) ETHOXY] METHYL]-7H-PYRROLO[2,3-D]PYRIMIDINE (5)

The above oily product of formula (16) was reacted with Aq.HCl (1.34 L) in tetrahydrofuran (378 mL) and DM water (1.5 L) at 25-30° C. Progress of the reaction was monitored by HPLC. After completion of the reaction, the reaction mass was treated with aq. Sodium carbonate and extracted with ethyl acetate and separated. The organic layer was washed with ˜10% w/v aqueous sodium chloride solution and separated. Finally, organic layer was treated with activated carbon, filtered through celite bed, concentrated under vacuum at below 45-50° C. to afford compound of formula (5) as crude product. Later, compound of formula (5) was further purified from acetonitrile followed by heptane and aqueous IPA to yield 4-(1H-pyrazol-4-yl)-7-[[2-(trimethylsilyl)ethoxy]methyl]-7H-pyrrolo[2,3-d]pyrimidine (308.8 g; 71.24% yield).

HPLC purity: >99.5%

### Example-07: PREPARATION OF 3-CYCLOPENTYL-3-[4-[7-(2-TRIMETHYLSILYL ETHOXYMETHYL)PYRROLO[2,3-D]PYRIMIDIN-4-YL]PYRAZOL-1-YL]PROPANE NITRILE (6)

Compound of formula (200 g; 5) was reacted with compound of formula (92.2 g; 4) in the presence of K₂CO₃ (8.8 g) in DMF (100 mL) at 25-30° C. Progress of the reaction was monitored by HPLC. After completion of the reaction, reaction mass was quenched with aqueous sodium chloride and extracted with MTBE. The organic layer was separated and distilled off completely under vacuum between 40-45° C. to afford 3-cyclopentyl-3-[4-[7-(2-trimethylsilylethoxymethyl)pyrrolo[2,3-d]pyrimidin-4-yl] pyrazol-1-yl]propanenitrile (6). Compound of formula (6) was used in the next stage as such without further purification.

### Example-08: PREPARATION OF (3R)-3-CYCLOPENTYL-3-[4-[7-(2-TRIMETHYLSILYLETHOXY METHYL)PYRROLO [2,3-D] PYRIMIDIN-4-YL] PYRAZOL-1-YL]PROPANENITRILE; (2S,3S)-2,3-DIBENZOYLOXY BUTANEDIOIC ACID (7a)

Compound of formula (6) was reacted with Di-Benzoyl-D-Tartaric acid (273 g; DBTA) in Acetonitrile and IPA mixture (3.2 L) at 75-80° C. followed by at 25-30° C. for 8-10 h. The same process is repeated twice to afford chirally enriched isomer of (3R)-3-cyclopentyl-3-[4-[7-(2-trimethylsilylethoxymethyl)pyrrolo[2,3-d]pyrimidin-4-yl] pyrazol-1-yl]propanenitrile; (2S,3S)-2,3-dibenzoyl oxybutanedioic acid (161.2 g; 7a).

HPLC purity: >99.5%

Chiral purity: >90.0% (by HPLC)

### Example-09: PREPARATION OF (βR)-B-CYCLOPENTYL-4-(7-(HYDROXYMETHYL)-7H-PYRROLO[2,3-D]PYRIMIDIN-4-YL)-1H-PYRAZOLE-1-PROPANENITRILE, (2S,3S)-2,3-BIS(BENZOYLOXY)-BUTANEDIOIC ACID (1.1) (10)

Compound of formula (150 g; 7a) in acetonitrile (750 mL) was selectively deprotected with BF₃·Etherate (64.4 g) at 25-30° C. Progress of the reaction was monitored by HPLC. After completion by HPLC, reaction mass proceeded as such for next stage without further isolation.

### Example-10: (βR)-B-CYCLOPENTYL-4-(7H-PYRROLO[2,3-D]PYRIMIDIN-4-YL)-1H-PYRAZOLE-1-PROPANENITRILE, (2S,3S)-2,3-BIS(BENZOYL OXY)-BUTANEDIOIC ACID (1.1) (17)

Above reaction mass containing (βR)-β-cyclopentyl-4-(7-(hydroxymethyl)-7H-pyrrolo[2,3-d]pyrimidin-4-yl)-1H-pyrazole-1-propanenitrile, (2S,3S)-2,3-bis(benzoyloxy)-butanedioic acid (1:1) (10) was reacted with Diethanolamine (198 g) at 25-30° C. Progress of the reaction was monitored by HPLC. After completion of reaction, reaction mass was quenched with DM water and extracted with ethyl acetate. The organic layer was separated and distilled off completely under vacuum between 45-50° C. The resulting oily residue dissolved in acetonitrile-IPA mixture (1.05 L) and was treated with DBTA (81.5 g) at 75-80° C. for 1 h followed by at 25-30° C. for 8-10 h and filtered. The wet compound was treated with aq. Sodium carbonate and extracted with ethyl acetate. The organic layer was separated and distilled to afford oily residue. Which was further treated with DBTA (68 g) in ACN:IPA mixture (1.0 L) at 55-60° C. for 1 h followed by at 25-30° C. for 8-10 h. The above salt breaking and making of DBTA salt repeated again to afford chirally and chemically pure (3R)-3-cyclopentyl-3-[4-(7H-pyrrolo[2,3-d]pyrimidin-4-yl)pyrazol-1-yl]propanenitrile (93.7 g; 74.79% by theory; 17).

HPLC purity: >99.5%

Chiral purity: >99.5% (by HPLC)

### Example-11: PREPARATION OF (R)-3-(4-(7H-PYRROLO[2,3-D]PYRIMIDIN-4-YL)-1H-PYRAZOL-1-YL)-3-CYCLOPENTYL PROPANENITRILE PHOSPHATE (1:1) (RUXOLITINIB PHOSPHATE; 1)

(βR)-β-cyclopentyl-4-(7H-pyrrolo[2,3-d]pyrimidin-4-yl)-1H-pyrazole-1-propanenitrile, (2S,3S)-2,3-bis (benzoyloxy)-butanedioic acid (1:1) (90.0 g; 17) was reacted with ˜85% orthophosphoric acid (24.0 g) in acetone (2.97 L) at 45-50° C. for 1 h followed by at 25-30° C. for 2 h and at 10-15° C. for 4 h, filtered, washed with acetone and dried to afford Ruxolitinib phosphate (51.8 g; 94.6% by theory).

HPLC purity: >99.5%

Chiral purity: >99.5% (by HPLC)

### Example-12: RECOVERY OF 4-(JH-PYRAZOL-4-YL)-7-[[2-(TRIMETHYLSILYL) ETHOXY] METHYL]-7H-PYRROLO[2,3-D]PYRIMIDINE (5) FROM MOTHER LIQUORS OF COMPOUND OF FORMULA (7a)

The mother liquors of compound of formula (˜13.8 L; 7a) containing majorly compound of formula (7b) were concentrated under vacuum. The resulting product was treated with aqueous sodium carbonate (144.8 g) and extracted with ethyl acetate. The organic layer was separated, washed with aq. sodium chloride, separated the organic layer and distilled. The resulting oily product was reacted with potassium tert. Butoxide (218.6 g) in acetonitrile (2.25 L) at 25-30° C. After completion of reaction monitored by HPLC, the reaction mixture was partitioned between ethyl acetate and water. Finally, organic layer was concentrated under vacuum at below 45-50° C. to afford compound of formula (5) as crude product. Later, compound of formula (5) was further purified from acetonitrile followed by heptane and aqueous IPA to yield 4-(1H-pyrazol-4-yl)-7-[[2-(trimethylsilyl) ethoxy]methyl]-7H-pyrrolo[2,3-d]pyrimidine (192.0 g; 71.83% yield).

HPLC purity: >99.5%

We claim:

1. A compound of Formula (14) having below structure:

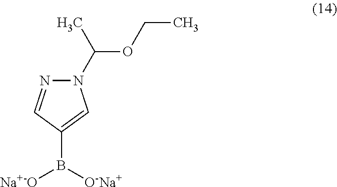

2. A process for preparation of novel compound of Formula (14) 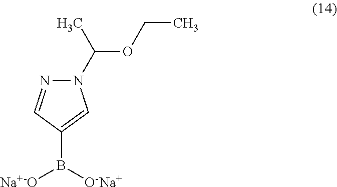
comprising the steps of:
a) reacting pyrazole of Formula (11) 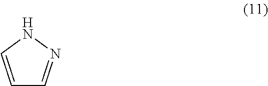
with bromine in an organic solvent to afford compound of Formula (12) 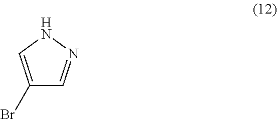
b) reacting the compound of Formula (12) with ethyl vinyl ether in an organic solvent in presence of catalytic amount of acid reagent to afford compound of formula (13) 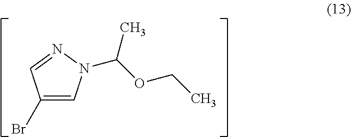
c) reacting the compound of formula (13) with triisopropylborate in presence of n-Butyl lithium in an organic solvent followed by reaction with aqueous sodium hydroxide to afford compound of formula (14).

3. The process as claimed in claim 2, wherein the organic solvent used in step a) is selected from dichloromethane, chloroform, acetonitrile or mixture thereof.

4. The process as claimed in claim 2, wherein,
In step-b), the acid catalyst used is gaseous HCl in combination with solvent selected from methanol, ethanol, ethyl acetate, 1,4-dioxane, isopropyl alcohol.
In step-b), the organic solvent used is selected from dichloromethane, chloroform, methanol, isopropylalchol, 1,4-dioxane, methyl t-butyl ether, tetrahydrofuran or mixture thereof,
In step-c), the organic solvent used is selected from tetrahydrofuran, 1,4-dioxane, methyl t-butyl ether, 2-methyl THF, cyclopentyl methyl ether or mixture thereof.

5. A process for the preparation of Ruxolitinib phosphate of Formula (1), 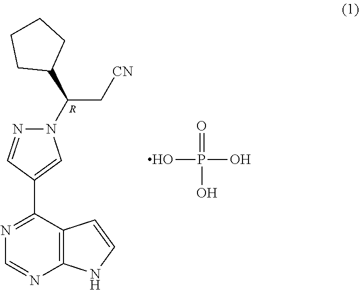
comprising the steps of:
a) reacting compound of formula (15) 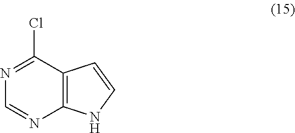
with SEM-chloride in the presence of a base in a solvent to afford compound of formula (9) 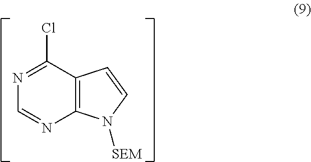
b) reacting the compound of formula (9) with novel compound of formula (14) 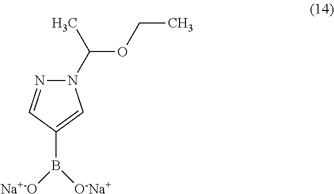
in the presence of palladium catalyst in an aqueous organic solvent to afford compound of formula (16), 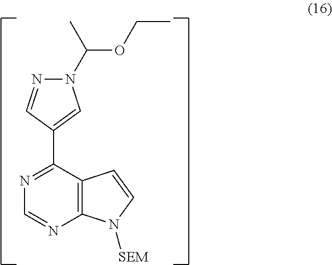
c) reacting the compound of formula (16) with an acid followed by treatment with aqueous base to afford compound of formula (5), 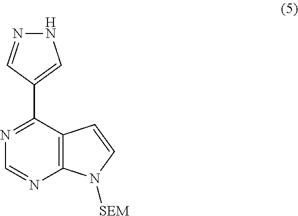 [SMILES: C[Si](C)(C)CCOCn1ccc2c(-c3cn[nH]c3)ncnc21]
d) reacting the compound of formula (5) with compound of formula (4) 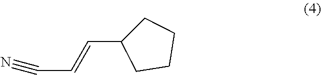
in the presence of a base in an organic solvent to afford compound of formula (6) 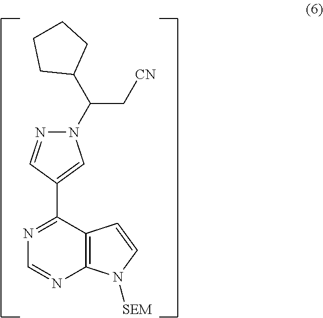
e) reacting the compound of formula (6) with Di-Benzoyl-D-Tartaric acid followed by crystallization in an organic solvent mixture to afford compound formula (7a) 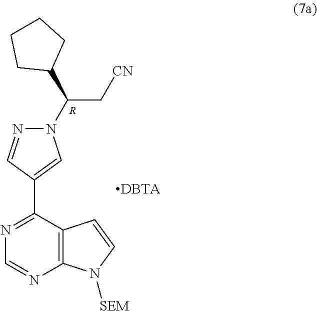
f) reacting the compound of formula (7a) with Boron trifluoride etherate in an organic solvent to afford compound of formula (10) 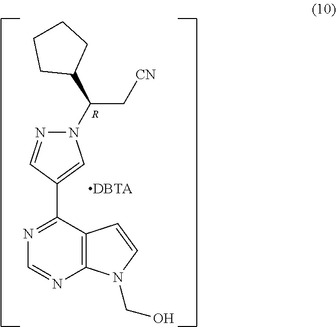
g) reacting the compound of formula (10) with an organic base followed by treatment with Di-Benzoyl-D-Tartaric acid and purification to afford compound of formula (17), 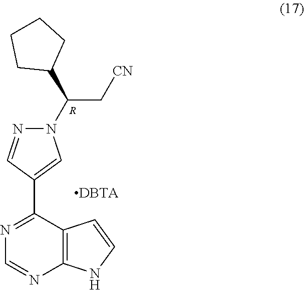
h) reacting the compound of formula (17) with phosphoric acid in an organic solvent to afford Ruxolitinib phosphate of formula (1),
i) purification of Ruxolitinib phosphate of formula (1) with an organic solvent or solvent mixture to afford chemically and chirally pure Ruxolitinib phosphate of formula (1).

6. The process as claimed in claim 5, wherein,
In step-a), the base used is selected from potassium carbonate, sodium carbonate, sodium hydride, cesium carbonate, potassium t-butoxide or mixture thereof.
In step-a), the organic solvent used is selected from N,N-dimethylformamide, N,N-dimethylacetamide, tetrahydrofuran, dimethylsulfoxide, N-Methylpyrrolidone, 1,4-dioxane or mixture thereof.

7. The process as claimed in claim 5, wherein,
In step-b) the palladium catalyst used is selected from tetrakis(triphenylphosphine)palladium (0), [1,1′-Bis(diphenylphosphino) ferrocene] dichloropalladium(II), [1,1′-Bis(diphenylphosphino) ferrocene] dichloro palladium(II) DCM complex, Bis(triphenylphosphine) palladium(II) dichloride, palladium acetate, palladium carbon;
In step-b) the solvent used is selected from 1,4-dioxane, tetrahydrofuran, water, isopropyl alcohol, ethanol, toluene, N,N-dimethylformamide, N,N-dimethylacetamide or mixture thereof.

8. The process as claimed in claim 5, wherein,
In step-c) the acid used is selected from aq. HCl, aq. acetic acid and the base used is selected from sodium hydroxide, potassium hydroxide, sodium carbonate and potassium carbonate.

9. The process as claimed in claim 5, wherein,
In step-d) the base used is selected from potassium carbonate, sodium carbonate, sodium hydride, cesium carbonate, potassium t-butoxide;
In step-d) the organic solvent used is selected from N,N-dimethylformamide, N,N-dimethylacetamide, tetrahydrofuran, dimethylsulfoxide, N-Methylpyrrolidone, 1,4-dioxane, methanol, ethanol, isopropyl alcohol.
In step-e) the organic solvent used is selected from isopropyl alcohol, acetonitrile, acetone, tetrahydrofuran, 1,4-dioxane, methanol, ethanol, dimethyl sulfoxide or mixture thereof.
In step-f) the organic solvent used is selected from ethyl acetate, methyl t-butyl ether, dichloromethane, chloroform, tetrahydrofuran, isopropyl alcohol, acetonitrile, acetone, tetrahydrofuran, 1,4-dioxane, methanol, ethanol, dimethyl sulfoxide.
In step-h) the organic solvent used is selected from acetone, acetonitrile, isopropyl alcohol, dichloromethane, tetrahydrofuran, 1,4-dioxane.

10. The process as claimed in claim 5, wherein in step-i) the organic solvent used is selected from acetone and isopropyl alcohol or mixture thereof.

11. A process for recovery of compound of formula (5) comprising steps of:
a) mother liquors of compound of formula (7a) containing majorly compound of formula (7b) is treating with aqueous base followed by extraction with an organic solvent 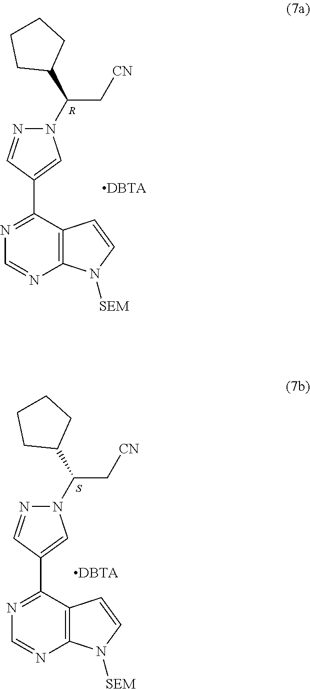
b) followed by treatment with base to afford compound of formula (5)

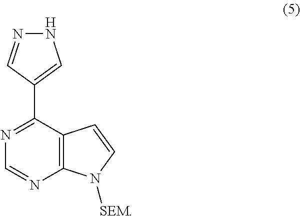 [SMILES: C[Si](C)(C)CCOCn1ccc2c(-c3cn[nH]c3)ncnc21]

12. The process as claimed in claim 11, wherein the compound of Formula (5) used in the process for the preparation of Ruxolitinib phosphate of formula (1).

13. The process as claimed in claim 11, wherein the base used in step a) and step b) is selected from aqueous potassium carbonate, aqueous sodium carbonate or aqueous sodium bicarbonate and solvent is selected from ethyl acetate or dichloromethane or methyl t-butyl ether or mixture thereof.
